(19) United States
(12) Patent Application Publication
(10) Pub. No.: US 2010/0121159 A1
(43) Pub. Date: May 13, 2010

(54) Devices and Methods for Monitoring Core Temperature and an Intraperitoneal Parameter

(76) Inventors: Daniel Rogers Burnett, San Francisco, CA (US); Amit Rajguru, Orinda, CA (US); Gregory W. Hall, Redwood City, CA (US); Christopher Hermanson, Santa Cruz, CA (US); Daniel Seidman, Oakland, CA (US)

Correspondence Address: SHAY GLENN LLP, 2755 CAMPUS DRIVE, SUITE 210, SAN MATEO, CA, 94403, US

(21) Appl. No.: 12/615,045

(22) Filed: Nov. 9, 2009

Related U.S. Application Data
(60) Provisional application No. 61/112,576, filed on Nov. 7, 2008.

(51) Int. Cl. A61B 5/01; A61B 5/00

(52) U.S. Cl. 600/301; 600/549

(57) ABSTRACT

Methods and apparatus for monitoring core temperature of a patient receiving intraperitoneal hypothermia or hyperthermia are provided which may include any number of features. One feature is placing a monitoring device having a sensor into a non-intraperitoneal cavity of a patient. The non-intraperitoneal cavity can be a urethra, a rectum, an anal sphincter, a stomach, an esophagus, the peripheral vasculature, or a vagina, for example. Another feature is sensing core temperature of the patient with the sensor.

### CROSS REFERENCE TO RELATED APPLICATIONS

This application claims the benefit under 35 U.S.C. 119 of U.S. Provisional Patent Application No. 61/112,576, filed Nov. 7, 2008, titled “Devices and Methods for Monitoring Core Temperature and an Intraperitoneal Parameter.” This application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates generally to medical/surgical devices and methods pertaining to hypothermia, hyperthermia and normothermia. More specifically, the present invention relates to devices and methods for monitoring core temperature and an intraperitoneal parameter such as pressure.

### BACKGROUND OF THE INVENTION

Hypothermia has been gaining credibility as a potential therapy for the treatment of a variety of diseases based on its cardio- and neuroprotective therapies. One of the difficulties with hypothermia therapy is that it is difficult, inconvenient, uncomfortable and/or painful to get accurate readings of the core temperature for open and/or closed loop control of the hypothermia treatment. Accuracy of these core temperature readings is critical however, particularly with rapid cooling therapies such as cold peritoneal lavage. With the rapid induction of hypothermia, overshoot (cooling a patient's temperature to too low a temperature) is a frequent problem and may cause fatal arrhythmias. Generating a reliable, accurate signal for the control of the hypothermic therapy provides for a safer, more effective therapeutic hypothermia therapy.

There are several obstacles to overcome with the measurement of core temperature during induction and maintenance of therapeutic hypothermia or normothermia. First of all access to true core temperature conventionally requires access to a cavity that is within or adjacent to the core. These cavities include the bladder, the rectum, the GI tract (e.g., esophagus or nasopharynx), and the tympanic membrane.

U.S. Pat. Nos. 4,413,633; 4,497,324; 6,602,243; and US Publication No. 2003/0114835 are all generally directed toward modified Foley catheters. They describe a Foley catheter with a temperature sensor adapted to measure the temperature of a patient's bladder and/or urine within the bladder. U.S. Pat. No. 5,335,669 in the name of Tihon et al. is generally directed toward a rectal probe with an inflatable, low pressure, compliant balloon having a temperature sensor associated with the wall thereof. The balloon is inflated to put the temperature element in intimate contact with the anterior side of the rectum.

Core temp measurement, however, is complicated within the bladder and rectum due to the variable presence of a thermal load (urine or stool) which may cause changes in the local temperature to lag drastically behind the actual core temperature and cause dangerous overshoot. In the time that it takes to cool the urine or stool down, the core temperature may already be in a dangerous range. In addition, for the preferred hypothermia method of peritoneal lavage, the bladder and rectum are within the peritoneal cavity, so the temperatures measured in this scenario may more accurately reflect the temperature of the lavage as opposed to the core temperature.

U.S. Pat. No. 5,249,585 in the name of Turner et al. is generally directed toward a urethral inserted applicator having a temperature sensor for measuring the temperature of the prostate tissue. A separable insulated temperature sensor is inserted in a flexible tube, attached exteriorly of the catheter, during treatment. This temperature sensor measures the temperature of the tissue surrounding the catheter (i.e. the prostate). However, Turner et al. do not describe measuring the temperature of the urethra in order to obtain an accurate core temperature measurement.

Measuring temperature via the esophagus, as described by Philips in U.S. Pat. No. 6,290,717 has several drawbacks. Phillips describes a disposable esophageal probe having dual temperature elements to provide redundancy in temperature feedback to a controller. These sensors however, are located at the distal end of the catheter, and therefore the accuracy of the temperature reading is highly dependent on the level of catheter insertion. Furthermore, measuring the temperature via the esophagus or airway can be quite uncomfortable and/or painful for the patient, and at times, it can be dangerous to obstruct the mouth or airway of the patient.

Temperature control using a tympanic membrane temperature sensor is unreliable as well, with a slight change in the angle of the sensor resulting in a dangerously variable reading. Measurement of the core temp has also been described with central venous catheters or hypothermia catheters placed within the inferior vena cava. These catheters require a major procedure to insert, though, and unless they reach the right heart, do not accurately reflect core temperature if they are sensing temperature in the same vessel that they are cooling.

Thus there is a need for devices and methods for monitoring parameters such as temperature and/or pressure that will not be influenced by the presence of a thermal load, the level of insertion of the sensor, or the method used for inducing therapeutic hypothermia. The present invention provides a new and useful solution for monitoring intraperitoneal parameters such as core temperature and pressure.

### SUMMARY OF THE INVENTION

The present invention relates generally to medical/surgical devices and methods to measure temperature. More specifically, the present invention relates to devices and methods for monitoring parameters such as core temperature and intraperitoneal pressure.

One aspect of the invention provides a device for monitoring core temperature of a patient receiving intraperitoneal hypothermia or hyperthermia. In some embodiments, the device includes a catheter that is inserted into a urethra of the patient and a sensor, coupled to the catheter, which functions to sense core temperature. In some embodiments, the catheter is inserted into an anal sphincter of the patient.

In some embodiments, the device further includes a distal retention element, coupled to a distal portion of the catheter, which functions to couple to a bladder and prevent the catheter from exiting the urethra. In some embodiments, the distal retention element functions to couple to a rectum and prevent the catheter from exiting the anal sphincter. In some embodiments the distal retention element is a balloon. In some embodiments, the sensor is coupled to the catheter, proximal to the distal retention element.

In some embodiments, the device further includes a proximal retention element, coupled to a proximal portion of the catheter, which functions to couple to an exterior portion of the patient and prevent the sensor from entering a bladder. In some embodiments, the proximal retention element functions to couple to an exterior portion of the patient and prevent the sensor from entering a rectum.

In some embodiments, the sensor includes a plurality of sensors that sense temperature at a plurality of locations within the urethra. In some embodiments, the plurality of sensors sense temperature at a plurality of locations within the anal sphincter. In some embodiments, the sensor includes a second sensor, coupled to the catheter, which functions to sense intraperitoneal pressure. In some embodiments, the second sensor is coupled to the distal retention element.

Another aspect of the invention provides a device for monitoring core temperature of a patient receiving intraperitoneal hypothermia or hyperthermia. In some embodiments, the device includes an airway management mechanism adapted to sample exhaled air from an airway of the patient and a sensor, coupled to the device, which functions to sense core temperature. In some embodiments, the airway management mechanism includes an endotracheal tube, a catheter, a bronchoscope, a facemask, or a ventilation tube. In some embodiments, the sensor functions to sense the temperature of exhaled air. In some embodiments, the sensor includes a plurality of sensors that function to sense temperature at a plurality of locations within the airway.

In some embodiments, the device further includes a distal retention element, coupled to a distal portion of the airway management mechanism, which functions to couple to the airway and prevent the airway management mechanism from moving. In some embodiments, the distal retention element is a balloon. In some embodiments, the sensor is coupled to the distal retention element.

Another aspect of the invention provides a device for monitoring core temperature of a patient receiving intraperitoneal hypothermia or hyperthermia. In some embodiments, the device includes a catheter that is inserted into a non-intraperitoneal structure of the patient; a plurality of sensors, coupled to the catheter, which functions to sense temperature at a plurality of locations within the non-intraperitoneal structure; and a processor, coupled to the plurality of sensors, which functions to determine core temperature from temperatures sensed by the plurality of sensors. In some embodiments, the catheter is adapted to be inserted into an esophagus, a urethra, or an anal sphincter,

In some embodiments, the processor further functions to select the portion of the temperatures sensed by the plurality of sensors that have the highest temperatures, while in some embodiments, the processor further functions to select the portion of the temperatures sensed by the plurality of sensors that have the lowest temperatures. In some embodiments, the processor further functions to select the portion of the temperatures sensed by the plurality of sensors that have a temperature that falls within a predetermined range.

In some embodiments, the device further includes a heating and cooling device, coupled to the processor, which functions to adjust the level of intraperitoneal hypothermia or hyperthermia given to the patient based on the core temperature determined by the processor. In some embodiments, the device further includes a pressure sensor, coupled to the catheter and the processor, which is inserted into an intraperitoneal structure of the patient. Access to the intraperitoneal structure could be through the abdominal wall, through the stomach wall (a natural orifice approach), or through the rectum wall, for example. In some embodiments, the pressure sensor is inserted into a stomach, bladder, or rectum.

Another aspect of the invention provides a method of monitoring core temperature of a patient receiving intraperitoneal hypothermia or hyperthermia. In some embodiments, the method includes the steps of placing a monitoring device having a sensor into a urethra of the patient and sensing core temperature with the sensor. In some embodiments, the method includes the step of placing a monitoring device having a sensor into an anal sphincter of the patient, and in some embodiments, the method includes the step of placing a monitoring device having a sensor into an airway of the patient and the step of sensing core temperature with the sensor includes sensing the temperature of exhaled air.

In some embodiments, the step of placing a monitoring device includes placing a plurality of sensors into the urethra of the patient and the step of sensing core temperature includes sensing temperature at a plurality of locations within the urethra. In some embodiments, the step of placing a monitoring device comprises placing a plurality of sensors into the anal sphincter of the patient and the step of sensing core temperature comprises sensing temperature at a plurality of locations within the anal sphincter. In some embodiments, the step of placing a monitoring device includes placing a plurality of sensors into the airway of the patient and the step of sensing core temperature includes sensing temperature at a plurality of locations within the airway.

In some embodiments, the step of placing a monitoring device having a sensor includes placing a monitoring device having a first sensor and a second sensor and the method further includes the steps of placing the second sensor into a bladder and sensing an intraperitoneal parameter with the second sensor. In some embodiments, the method further includes the step of placing the second sensor into a rectum. In some embodiments, the step of sensing the intraperitoneal parameter includes sensing pressure.

Another aspect of the invention provides a method of monitoring core temperature of a patient receiving intraperitoneal hypothermia or hyperthermia. In some embodiments, the method includes the steps of placing a monitoring device having a plurality of sensors into a non-intraperitoneal structure of the patient, sensing temperature with the plurality of sensors, and determining core temperature from a portion of the temperatures sensed by the plurality of sensors. In some embodiments, the step of determining core temperature includes selecting a portion of the temperatures sensed by the plurality of sensors. In some embodiments, the step of determining core temperature includes selecting the highest sensed temperature, while in some embodiments, the step of determining core temperature includes selecting the lowest sensed temperature. In some embodiments, the step of determining core temperature includes selecting temperatures sensed by the plurality of sensors that fall within a predetermined range.

In some embodiments, the step of placing a monitoring device includes placing a monitoring device into an esophagus and the step of sensing temperature includes sensing a temperature of the esophagus at a plurality of locations along the esophagus. In some embodiments, the step of placing a monitoring device includes placing a monitoring device into a urethra and the step of sensing temperature includes sensing a temperature of the urethra at a plurality of locations along the urethra. In some embodiments, the step of placing a monitoring device includes placing a monitoring device into an anal sphincter and the step of sensing temperature includes sensing a temperature of the anal sphincter at a plurality of locations along the anal sphincter.

In some embodiments, the method further includes the step of adjusting the level of intraperitoneal hypothermia or hyperthermia given to the patient based on the core temperature determined from a portion of the temperatures sensed by the plurality of sensors. In some embodiments, the method further includes the steps of placing a sensor into an intraperitoneal structure, sensing a parameter with the sensor, and determining an intraperitoneal parameter from the parameter sensed by the sensor in the intraperitoneal structure. In some embodiments, the step of determining the intraperitoneal parameter includes determining pressure.

In some embodiments, the step of placing a monitoring device includes placing a monitoring device into an esophagus and the step of placing a sensor into an intraperitoneal structure includes placing a sensor into a stomach. In some embodiments, the step of placing a monitoring device includes placing a monitoring device into a urethra and the step of placing a sensor into an intraperitoneal structure includes placing a sensor into a bladder. In some embodiments, the step of placing a monitoring device includes placing a monitoring device into an anal sphincter and the step of placing a sensor into an intraperitoneal structure includes placing a sensor into a rectum.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a monitoring device having a sensor in a non-intraperitoneal structure according to certain embodiments.

FIGS. 2A-3B show a method of monitoring core temperature according to certain embodiments.

FIGS. 4A-4B show a monitoring device in a urethra according to certain embodiments.

FIGS. 5A-5B show a monitoring device in an anal sphincter according to certain embodiments.

FIGS. 6A-6B show a monitoring device in an esophagus according to certain embodiments.

FIG. 7 shows a monitoring device in an airway according to certain embodiments.

FIG. 8 shows a monitoring device in peripheral vasculature according to certain embodiments.

### DETAILED DESCRIPTION OF THE INVENTION

Various embodiments of devices and systems, as well as methods for monitoring core temperature of a patient receiving intraperitoneal hypothermia or hyperthermia. The following description is not intended to limit the invention to these embodiments, but rather to enable any person skilled in the art to make and use this invention.

The devices and methods discussed herein generally describe monitoring core temperature of a patient receiving intraperitoneal hypothermia or hyperthermia treatment. Systems and methods for inducing therapeutic hypothermia in a patient through the intraperitoneal cavity are discussed in commonly owned U.S. patent application Ser. Nos. 10/523,857, 11/552,090, 12/098,365, and 12/169,566, which are incorporated herein by reference. In some embodiments, therapeutic hypothermia is induced with a peritoneal lavage in the patient.

### 1. Devices for Monitoring Core Temperature

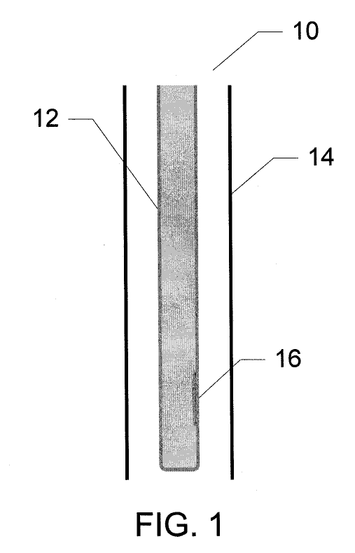

As shown in FIG. 1, the device 10 for monitoring core temperature of a patient receiving intraperitoneal hypothermia or hyperthermia includes a catheter 12 that is inserted into a non-intraperitoneal structure 14 of the patient and a temperature sensor 16 coupled to the catheter 12. The monitoring device is designed to monitor the core temperature of the patient while the patient receives intraperitoneal hypothermia or hyperthermia. The device may be alternatively used to monitor any other suitable parameter of a patient receiving any other suitable therapy, in any suitable environment, and for any suitable reason.

The catheter is inserted into a non-intraperitoneal structure of the patient. The catheter functions to position the sensor within the non-intraperitoneal structure such that the sensor can adequately sense core temperature from within the non-intraperitoneal structure. In some embodiments, the catheter further functions to act as a conduit for fluids, solids, or gasses leaving or entering the body of a patient. In some embodiments, the catheter is made from a flexible, biocompatible material such as latex, but may alternatively be made of any other suitable material such as other polymers or metal. The catheter may be one of several variations.

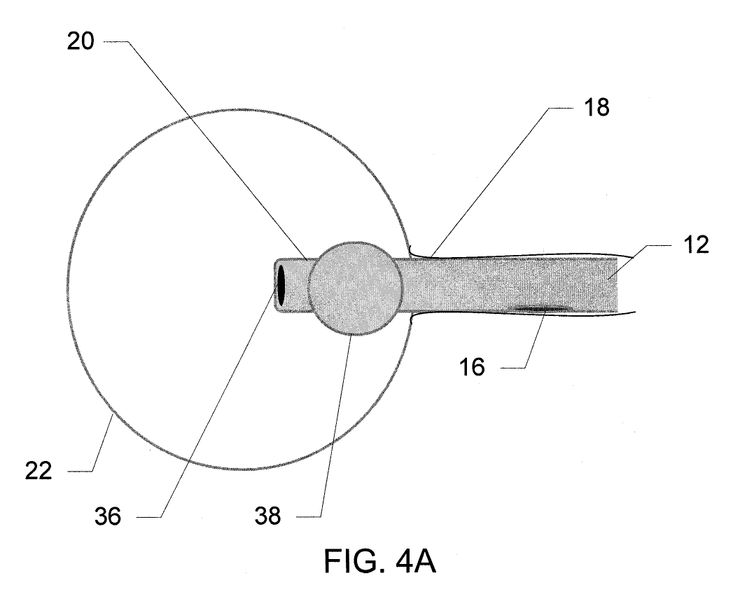

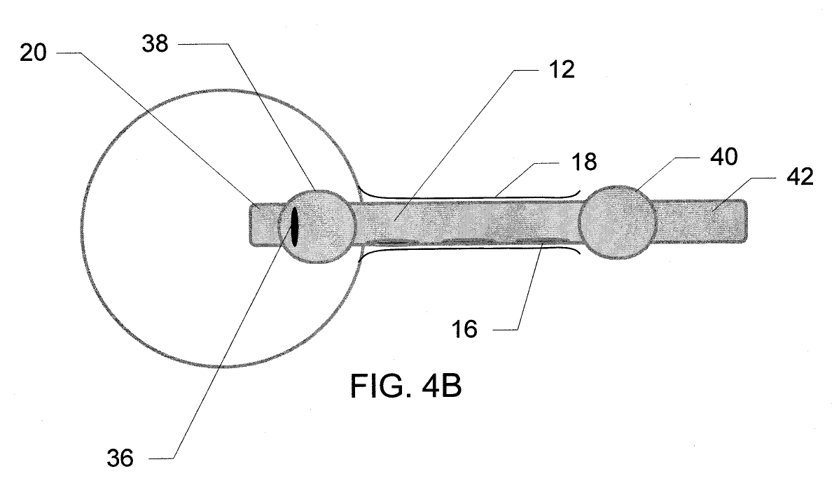

In a first variation, as shown in FIGS. 4A and 4B, the catheter 12 is a “Foley”-type catheter. In this variation, the non-intraperitoneal structure is a urethra 18, and the catheter functions to position the sensor 16 within the urethra and outside of the bladder 22, such that the sensor can adequately sense core temperature from within the urethra. This positioning provides for accurate temperature sensing during peritoneal cooling or heating. The bladder is an intraperitoneal structure, and therefore, its temperature will not represent the core temperature of the patient when hypo- or hyperthermia is received by the patient through peritoneal lavage. The urethra, on the other hand, is not an intraperitoneal structure. Furthermore, this positioning may also prevent the thermal load of any urine present within the bladder from slowing down the response or sensing time of the sensor. As shown in FIGS. 4A and 4B, the catheter of this variation, may have a distal portion 20 which may be inserted into the bladder. The catheter of this variation may additionally function to drain urine from the bladder.

In one embodiment, when the temperature sensor is positioned in a urethra, it is thermally insulated from the flow of colder urine. Because the bladder is an intraperitoneal structure, urine is cooled rapidly during rapid induction into hypothermia so as to cause the urethral temperature to not track as well with core temperature. Thus, in some embodiments, initial core temperature measurements can be monitored with a temperature sensor in another, non-urethral non-intraperitoneal structure during hypothermia inducement, and then the temperature measurement can be changed to urethral measurement mid-treatment.

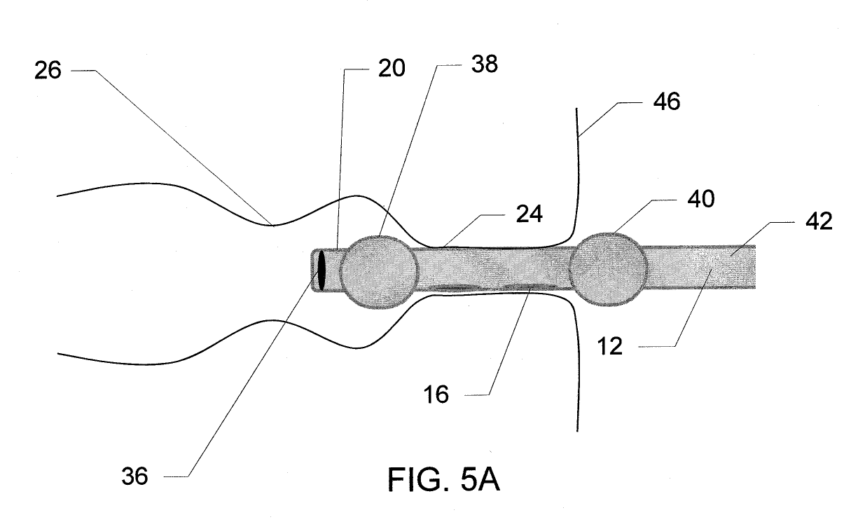

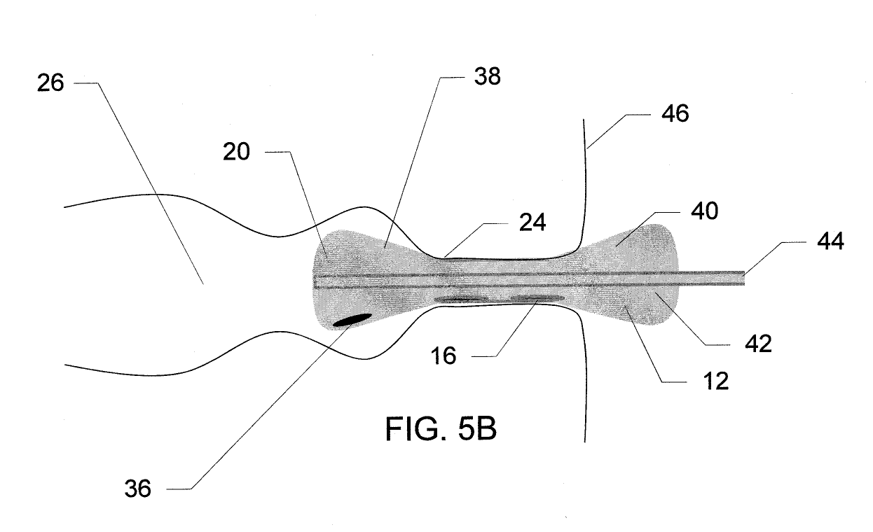

In a second variation, as shown in FIGS. 5A and 5B, the catheter 12 has been designed and sized to be inserted into an anal sphincter. In this variation, the non-intraperitoneal structure is an anal sphincter 24, and the catheter functions to position the sensor 16 within the anal sphincter and outside of the rectum 26, such that the sensor can adequately sense core temperature from within the anal sphincter. This positioning provides for accurate parameter sensing during peritoneal cooling or heating. The rectum is an intraperitoneal structure, and therefore, its temperature will not represent the core temperature of the patient when hypo- or hyperthermia is received by the patient through peritoneal lavage. The anal sphincter, on the other hand, is not an intraperitoneal structure. Furthermore, this positioning in the anal sphincter, outside of the rectum may also prevent the thermal load of any fecal matter present within the rectum from slowing down the response or sensing time of the sensor. As shown in FIGS. 5A and 5B, the catheter of this variation may have a distal portion 20 which may be inserted into the rectum. The catheter of this variation may additionally function to remove fecal matter from the rectum.

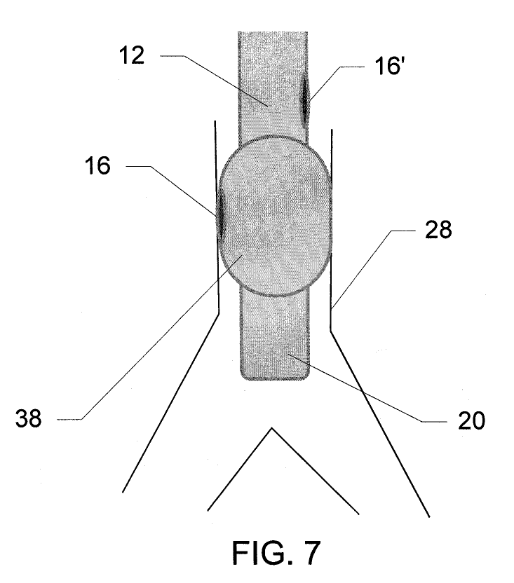

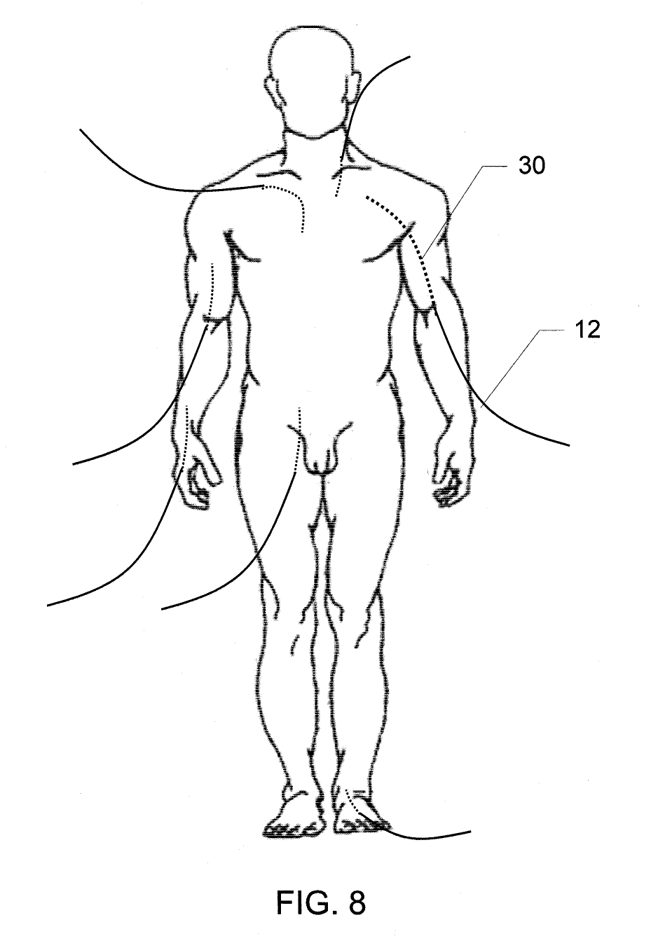

In a third variation, as shown in FIG. 7, the catheter 12 is an endotracheal tube. In this variation, the non-intraperitoneal structure is an airway 28, and the catheter functions to position the sensor 16 within the airway and facing externally towards the tracheal mucosa, such that the sensor can adequately sense temperature from within the airway. This positioning provides for accurate parameter sensing during peritoneal cooling or heating by positioning the sensor such that it can measure a parameter of air, exhaled by the lungs and exiting through the airway. In some embodiments, the catheter functions to position an additional sensor 16′ internally facing within the catheter, such that the sensor 16′ can measure a parameter of air, inhaled by the lungs and entering through the airway. The measured parameter, may then be extrapolated based on the difference between inhaled and exhaled air, which may be a particularly useful method in a controlled setting (steady ambient temperature) and for maintenance of hypo- or hyperthermia therapy following induction. As shown in FIG. 7, the catheter of this variation may have a distal portion 20, which may be inserted into the airway, adjacent to the lungs. The catheter of this variation may additionally function to allow air (or any other suitable fluids or gasses) into or out of the lungs. In an alternative version, a sensor may be positioned on a catheter or other suitable device (such as a face mask or a ventilation tube) on the exterior of a patient and measure a parameter of air, exhaled by the lungs and exiting through the mouth or nose.

In a fourth variation, as shown in FIG. 8, the catheter 12 has been designed and sized to be inserted into the peripheral vasculature. The catheter of this variation may be inserted through the skin into a peripheral vein 30. A peripheral vein may be any vein that is not in the chest or abdomen, such as a vein of the arm or leg. In this variation, the non-intraperitoneal structure is a peripheral vein, and the catheter functions to position the sensor (not shown) within the peripheral vasculature, such that the sensor can adequately sense core temperature from within the peripheral vasculature. This positioning provides for accurate parameter sensing during peritoneal cooling or heating. As shown in FIG. 8, there are several potential sites for insertion of the catheter including the internal jugular, the subclavian, the femoral, the antecubital fossa and/or the hand or foot, or any other suitable vascular site. While traditionally it has been felt that a central catheter is required for accurate core temperature, core temperature is accurately represented by the peripheral vasculature, particularly in the setting of hypothermia where the distal vasculature may clamp down and shunt blood back to the core more rapidly. The catheter of this variation may be a functional, peripherally inserted catheter, such as a PICC, an infusion catheter, a standard peripheral catheter, etc. The catheter may allow the passage of blood, drugs, or any other suitable fluids into or out of the vasculature.

In yet another variation (not shown), the catheter may be designed to be inserted into a patient's vagina to monitor core temperature.

The sensor of the device for monitoring core temperature is coupled to the catheter. In some embodiments, the sensor is a non-contact sensor, i.e. a sensor which is not required to come in contact with an object in order to detect a parameter of the object. The non-contact sensor may be an Infrared (IR) sensor, an optical pyrometer, a fiber optic thermometer, an acoustic meter, an ultrasonic meter, or any other suitable, non-contact means of detecting temperature or any other suitable parameter such as pressure. Alternatively, in some embodiments, the sensor is a thermometer, a thermocouple, a thermistor and/or an RTD (resistance temperature detector) to measure temperature and/or an altimeter, barometer, barograph, or a pressure gauge to measure pressure. In some embodiments the sensor may be a fluid filled balloon that functions to measure pressure. The fluid may be air, water, or any other suitable fluid. Intraperitoneal pressure measurement catheters may have depth markings to enable the user to determine the location of the pressure sensor within the cavity into which the catheter has been inserted. Such pressure measurement catheters may also have anchors to help position, and maintain the position, of the catheter and sensor. The sensor may alternatively be any other suitable sensor to measure any other suitable parameter in any suitable combination. In one embodiment, the catheter uses an external pressure sensor connected to the patient with a fluid column.

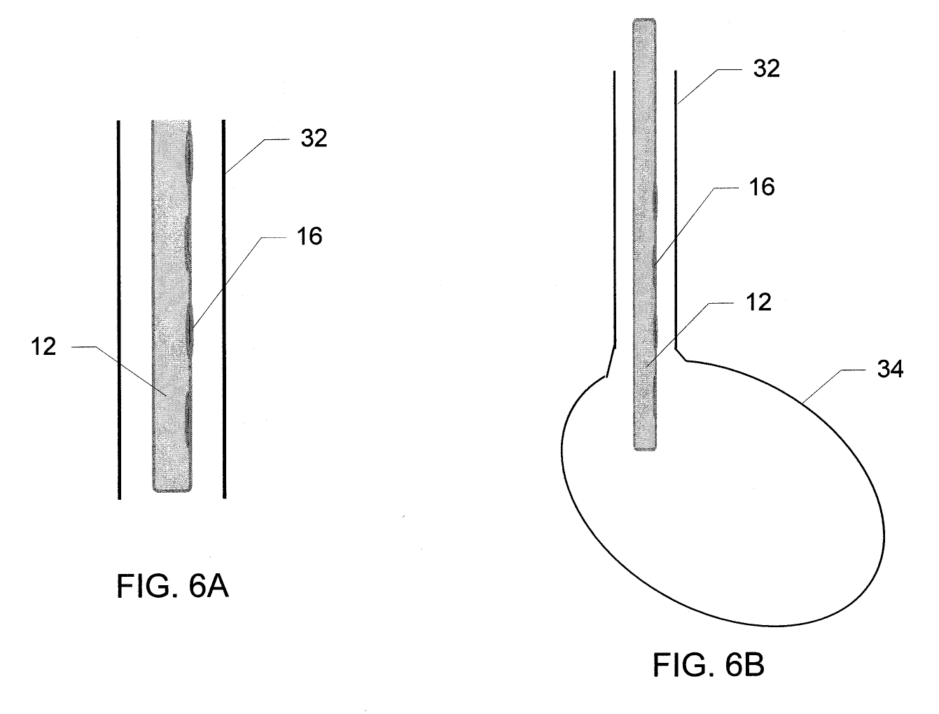

In some embodiments, the sensor includes a plurality of sensors, coupled to the catheter, that function to sense temperature at a plurality of locations within the non-intraperitoneal structure. The plurality of sensors may be scattered along the length of the catheter or may alternatively be arranged in a linear or helical fashion. Additionally, there may be one or more sensors at each location along the catheter to provide redundancy at every location. The plurality of sensors function to provide redundant parameter measurement. This redundancy helps to ensure the accuracy and/or precision of the measurement of the parameter. The plurality of sensors further function to sense the parameter at different locations (i.e. depths of insertion) along the catheter so that at least one of the sensors will be adjacent to, or in contact with the desired location or structure. An erroneously inserted sensor (under inserted or over inserted, i.e. over inserted into an intraperitoneal structure) will not agree with the other sensors. The plurality of sensors may be configured in one of several variations. In a first variation, as shown in FIG. 4B, the catheter 12 functions to position the plurality of sensors 16 (three sensors shown) within the urethra 18 and outside of the bladder 22, such that the plurality of sensors can adequately sense temperature from within the urethra. In a second variation, as shown in FIGS. 5A and 5B, the catheter 12 functions to position the plurality of sensors 16 (two sensors shown) within the anal sphincter and outside of the rectum 26, such that the sensor can adequately sense temperature from within the anal sphincter. In a third variation, as shown in FIGS. 6A and 6B, the catheter 12 functions to position the plurality of sensors 16 (four sensors shown) within the esophagus 32 and outside of the stomach 34, such that the plurality of sensors can adequately sense temperature from within the esophagus. In some embodiments, the plurality of sensors may be coupled to the catheter such that they span the entire length of the catheter and/or are located in both a non-intraperitoneal structure and an intraperitoneal structure. In one embodiment, the catheter could have temperature sensors in the esophagus and a pressure sensor near the distal end of the catheter that could then be positioned in the stomach and/or the peritoneal cavity.

In some embodiments, the plurality of sensors may be coupled to a processor or any other suitable mechanism for determining core temperature from a portion of the plurality of sensed parameters at the plurality of locations within the non-intraperitoneal structure. In a first variation, the processor functions to select the portion of the parameters sensed by the plurality of sensors having a parameter that falls within a predetermined range. The processor may select the optimal sensed parameter response to heating or cooling therapy, based on a predetermined optimal response. The optimal response may be based on known or predicted responses by non-intraperitoneal structures (such as a urethra, an anal sphincter, or an esophagus) to the cooling or heating therapy. In a second variation, the processor may select the optimal sensed parameter by ignoring inaccurate sensed parameters and/or selecting the sensors that are acting most appropriately. For example, slow response sensors may be ignored as they are likely located too superior (in the nasopharynx or exterior to the patient, for example) or located too inferior (in the stomach, bladder, or rectum, for example). Also, for example, sensed parameters that begin at temperatures (or pressures) above or below body temperature (or pressure) may be ignored as they are likely in the pharynx, upper esophagus, or exterior to the patient and may represent the impact of ambient air. In a third variation, the processor functions to select the portion of the parameters sensed by the plurality of sensors having the highest sensed parameters. In a fourth variation, the processor functions to select the portion of the parameters sensed by the plurality of sensors having the lowest sensed parameters.

The processor may further function to control the intensity of the intraperitoneal hypothermia or hyperthermia therapy given to the patient by being coupled to a heating/cooling device that functions to provide intraperitoneal hypothermia or hyperthermia to a patient. For example, if the intraperitoneal parameter, such as core temperature, is too high or too low, the processor may automatically adjust the therapy until a correct parameter level has been reached. Alternatively, the processor may simply indicate the sensed parameter through a display or any other suitable apparatus.

In some embodiments, the device for monitoring an intraperitoneal parameter of a patient receiving intraperitoneal hypothermia or hyperthermia further includes a second sensor 36, as shown in FIGS. 4A-5B. The second sensor is coupled to the catheter 12 and functions to sense an intraperitoneal parameter. The second sensor is inserted into an intraperitoneal structure of the patient, such as a bladder 22 (FIGS. 4A and 4B) or rectum 26 (FIGS. 5A and 5B). The second sensor is coupled distal portion 20 of the catheter, and in some embodiments, is coupled to the distal retention element (described below). In some embodiments, the intraperitoneal parameter sensed by the second sensor is the pressure of the peritoneal cavity, measured through the bladder, rectum, stomach, etc. Alternatively, it may be any other suitable intraperitoneal parameter such as temperature.

In some embodiments, the device for monitoring core temperature of a patient receiving intraperitoneal hypothermia or hyperthermia further includes a distal retention element 38, as shown in FIGS. 4A-5B and 7. In some embodiments, the distal retention element is a balloon, but may alternatively be a loop, a pigtail, a malecot or any other suitable retention element. The distal retention element may be selectively engaged and disengaged. For example, the distal retention element may be inflatable. Air or fluid, such as saline, may be inserted and/or removed through a lumen 44, as shown in FIG. 5B, in order to selectively engage and disengage the distal retention element. In some embodiments, the sensor or plurality of sensors, are coupled to the catheter, proximal to (i.e. toward the proximal end from) the distal retention element. The distal retention element may be configured in one of several variations. In a first variation, as shown in FIGS. 4A and 4B, the distal retention element 38 is coupled to the distal portion 20 of the catheter 12 and functions to couple to a bladder 22 and prevent the catheter from exiting the urethra 18. In a second variation, as shown in FIGS. 5A and 5B, the distal retention element 38 is coupled to the distal portion 20 of the catheter 12 and functions to couple to a rectum 26 and prevent the catheter from exiting the anal sphincter 24. In a third variation, as shown in FIG. 7, the distal retention element is coupled to the distal portion 20 of the catheter 12 and functions to couple to the airway 28 and prevent the catheter from entering the lungs and may additionally function to provide an airtight seal within the airway.

In some embodiments, the device for monitoring core temperature of a patient receiving intraperitoneal hypothermia or hyperthermia further includes a proximal retention element 40, as shown in FIGS. 4B-5B. In some embodiments, the proximal retention element is a balloon, but may alternatively be a loop, a pigtail, a malecot, a sliding cuff, adhesive, sutures, a sliding circumferential clamp (e.g., Touhy-Borst valve) or any other suitable retention element. The proximal retention element may be selectively engaged and disengaged. For example, the proximal retention element may be inflatable. Air or fluid, such as saline, may be inserted and/or removed through a lumen 44, as shown in FIG. 5B, in order to selectively engage and disengage the proximal retention element. The proximal retention element may be configured in one of several variations. In a first variation, as shown in FIG. 4B, the proximal retention element 38 is coupled to a proximal portion 42 of the catheter 12 and functions to couple to an exterior portion of the patient and prevent the sensor from entering a bladder. In a second variation, as shown in FIGS. 5A and 5B, the proximal retention element 38 is coupled to the proximal portion 42 of the catheter 12 and functions to couple to an exterior portion of the patient 46 and prevent the sensor from entering a rectum 26. In some embodiments, as shown in FIG. 5B, the distal and proximal retention elements may include a saddle-shaped balloon. The saddle-shaped balloon may include a single balloon or multiple balloons. In the case of a single balloon, the proximal and distal retention elements may inflate and deflate at different pressures allowing the proximal and/or distal retention element to be inflated, the device to be positioned, and then the remaining retention element to be inflated using the same inflation lumen.

### 2. Methods of Monitoring an Intraperitoneal Parameter

A method of monitoring core temperature of a patient receiving intraperitoneal hypothermia or hyperthermia generally includes inducing therapeutic hypothermia or hyperthermia in a patient. Therapeutic hypothermia or hyperthermia can be induced by gaining access to a peritoneal cavity with an access device, such as with a trocar or cannula, inserting a catheter into the peritoneal cavity through the access device, and lavaging the cavity with a therapeutic fluid to induce hypothermia or hyperthermia. The fluid can be chilled, for example, to induce hypothermia or warmed to induce hyperthermia.

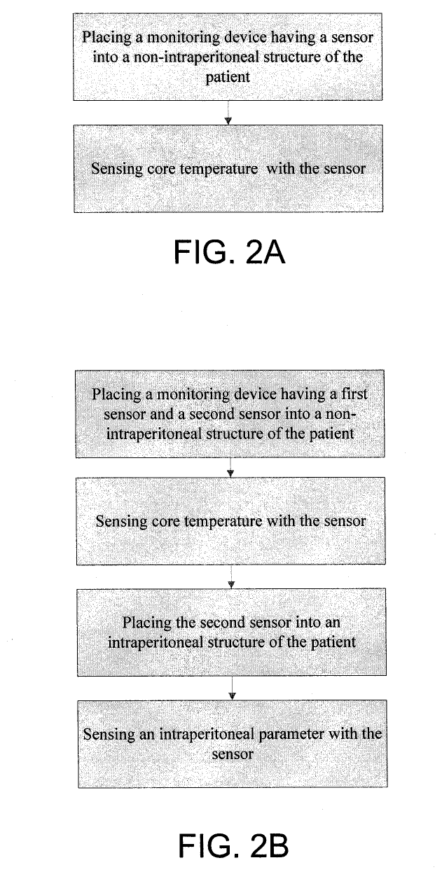

As shown in FIG. 2A, the method can further include the step of placing a monitoring device having a sensor into a non-intraperitoneal structure of the patient and sensing core temperature with the sensor. The method may be alternatively used for monitoring any other suitable parameter of a patient receiving any other suitable therapy, in any suitable environment, and for any suitable reason.

The step of placing a monitoring device having a sensor into a non-intraperitoneal structure of the patient, functions to position the monitoring device into a non-intraperitoneal structure such as a urethra 18 (FIG. 4A), an anal sphincter 24 (FIG. 5A), an esophagus 32 (FIG. 6A), an airway 28 (FIG. 7), or peripheral vasculature 30 (FIG. 8). In some embodiments, the step of placing a monitoring device comprises placing a plurality of sensors into the non-intraperitoneal structure of the patient.

The step of sensing core temperature with the sensor, functions to sense core temperature from within a non-intraperitoneal structure. In some embodiments, the step of sensing core temperature with the sensor comprises the steps of sensing the temperature of the non-intraperitoneal structure (such as the temperature of the urethra, the anal sphincter, or exhaled air within the airway) and thereby sensing the core temperature of the patient. In some embodiments, where the step of placing a monitoring device comprises placing a plurality of sensors into the non-intraperitoneal structure of the patient, the step of sensing core temperature comprises sensing temperature at a plurality of locations within the non-intraperitoneal structure.

As shown in FIG. 2B, the method of monitoring core temperature of a patient receiving intraperitoneal hypothermia or hyperthermia includes the steps of placing a monitoring device, having a first sensor and a second sensor, into a non-intraperitoneal structure of the patient; placing the second sensor into an intraperitoneal structure of the patient; sensing core temperature with the first sensor; and sensing an intraperitoneal parameter, such as pressure, with the second sensor.

The step of placing a monitoring device, having a first sensor and a second sensor, into a non-intraperitoneal structure of the patient, functions to position the monitoring device and the first sensor into a non-intraperitoneal structure such as a urethra 18 (FIG. 4A), an anal sphincter 24 (FIG. 5A), or an esophagus 32 (FIG. 6A). The step of placing the second sensor into an intraperitoneal structure of the patient, functions to position the second sensor into an intraperitoneal structure such as a bladder 22 (FIG. 4A), a rectum 26 (FIG. 5A), or a stomach 34 (FIG. 6B).

The step of sensing core temperature with the first sensor, functions to sense core temperature from within a non-intraperitoneal structure. In some embodiments, the step of sensing core temperature with the sensor comprises the steps of sensing the temperature of the non-intraperitoneal structure (such as the temperature of the urethra, the anal sphincter, or exhaled air within the airway) and therefore sensing the core temperature of the patient. In some embodiments, there is an algorithmic relationship between the sensed temperature and the core temperature of the patient. For example, the core temperature may be calculated by taking the average of all sensed temperatures, the average of the warmest two sensed temperatures, etc.

The step of sensing an intraperitoneal parameter with the second sensor, functions to sense an intraperitoneal parameter from within an intraperitoneal structure. In some embodiments, the step of sensing the intraperitoneal parameter with the sensor comprises the steps of sensing the pressure of the intraperitoneal structure (such as the pressure of the bladder, rectum, or stomach) and therefore sensing the intraperitoneal pressure of the patient.

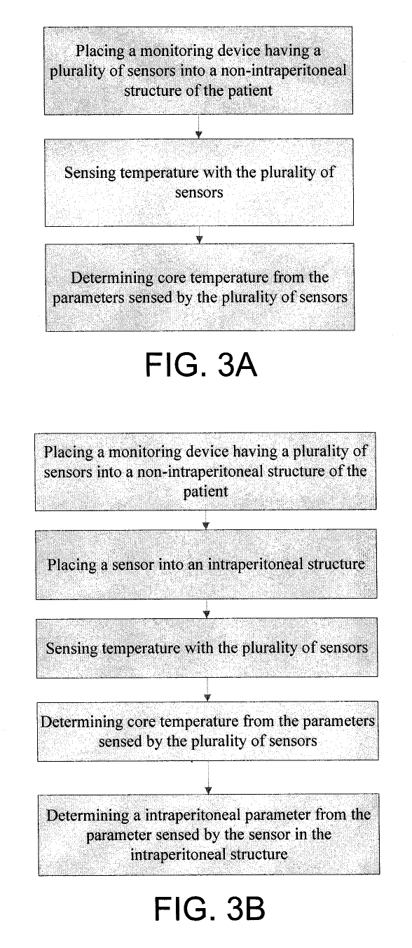

As shown in FIG. 3A, in some embodiments, the method of monitoring core temperature of a patient receiving intraperitoneal hypothermia or hyperthermia includes the steps of placing a monitoring device having a plurality of sensors into a non-intraperitoneal structure of the patient, sensing temperature with the plurality of sensors, and determining core temperature from a portion of the temperatures sensed by the plurality of sensors. The method may be alternatively used for monitoring any other suitable parameter of a patient receiving any other suitable therapy, in any suitable environment, and for any suitable reason.

In some embodiments, the step of sensing temperature comprises the step of sensing the temperature of the non-intraperitoneal structure (such as the temperature of the urethra, the anal sphincter, or exhaled air within the airway) at a plurality of locations (within and around the non-intraperitoneal structure). The step of determining core temperature from a portion of the temperatures sensed by the plurality of sensors, functions to obtain the core temperature from a series of sensed parameters. In some variations, the step of determining core temperature comprises selecting a portion of the temperatures sensed by the plurality of sensors, selecting the highest sensed parameter, selecting the lowest sensed parameter, selecting a sensed temperature that falls within a predetermined range, and/or any suitable combination thereof.

In some embodiments, the method of monitoring core temperature of a patient receiving intraperitoneal hypothermia or hyperthermia further includes the step of further adjusting the level of intraperitoneal hypothermia or hyperthermia given to the patient based on the intraperitoneal parameter determined from a portion of the parameters sensed by the plurality of sensors.

In some embodiments, as shown in FIG. 3B, the method of monitoring core temperature of a patient receiving intraperitoneal hypothermia or hyperthermia further includes the steps of placing a sensor into an intraperitoneal structure, sensing a parameter with the sensor, and determining an intraperitoneal parameter, such as pressure, from the sensor in the intraperitoneal structure. As described above, the step of placing the sensor into an intraperitoneal structure of the patient, functions to position the sensor into an intraperitoneal structure such as a bladder 22 (FIG. 4A), a rectum 26 (FIG. 5A), or a stomach 34 (FIG. 6B). The step of sensing a parameter with the second sensor, functions to sense a parameter from within an intraperitoneal structure. In some embodiments, this step comprises the step of sensing the pressure of the intraperitoneal structure (such as the pressure of the bladder, rectum, or stomach), and the step of determining an intraperitoneal parameter from the parameter sensed by the sensor in the intraperitoneal structure comprises determining the intraperitoneal pressure of the patient.

In addition, the methods described herein may be applied to many of the devices and systems described in any of the reference listed below. In particular, these references describe devices, systems, and methods for providing hypothermia to a patient and/or monitoring a patient receiving hypothermia. Thus, the following patents/patent applications are herein incorporated by reference in their entirety:

| TITLE | US Appln. No. | Filing Date |
|---|---|---|
| MEDICAL DEVICE FOR THE EXTRAVASCULAR | 10/523,857 | May 11, 2005 |
| RECIRCULATION OF FLUID IN BODY CAVITIES | | |
| AT CONTROLLED TEMPERATURE AND PRESSURE | | |
| METHOD AND APPARATUS FOR PERITONEAL | 11/552,090 | Oct. 23, 2006 |
| HYPOTHERMIA AND/OR RESUSCITATION | | |
| DEVICE AND METHOD FOR SAFE ACCESS TO A | 12/098,355 | Apr. 4, 2008 |
| BODY CAVITY | | |
| HYPOTHERMIA DEVICES AND METHODS | 12/169,566 | Jul. 8, 2008 |

As a person skilled in the art will recognize from the previous detailed description and from the figures and claims, modifications and changes can be made to the embodiments of the invention without departing from the scope of this invention defined in the following claims. For example, redundant temperature and/or pressure sensors may be used in the embodiments described above for added safety.

What is claimed is:

1. A device for monitoring core temperature of a patient receiving intraperitoneal hypothermia or hyperthermia, the device comprising:
a catheter adapted to be inserted into a urethra of the patient;
a sensor coupled to the catheter and adapted to sense core temperature.

2. The device of claim 1, further comprising a distal retention element, coupled to a distal portion of the catheter and adapted to couple to a bladder and prevent the catheter from exiting the urethra.

3. The device of claim 2, wherein the distal retention element is a balloon.

4. The device of claim 2, wherein the sensor is coupled to the catheter, proximal to the distal retention element.

5. The device of claim 1, further comprising a proximal retention element, coupled to a proximal portion of the catheter and adapted to couple to an exterior portion of the patient and prevent the sensor from entering a bladder.

6. The device of claim 1, wherein the sensor comprises a plurality of sensors adapted to sense temperature at a plurality of locations within the urethra.

7. The device of claim 1, further comprising a second sensor coupled to the catheter and adapted to sense intraperitoneal pressure.

8. The device of claim 7, further comprising a distal retention element, coupled to a distal portion of the catheter and adapted to couple to a bladder and prevent the catheter from exiting the urethra, and wherein the sensor is coupled to the catheter, proximal to the distal retention element and the second sensor is coupled to the distal retention element.

9. A device for monitoring core temperature of a patient receiving intraperitoneal hypothermia or hyperthermia, the device comprising:
a catheter adapted to be inserted into an anal sphincter of the patient;
a sensor coupled to the catheter and adapted to sense core temperature.

10. The device of claim 9, further comprising a distal retention element, coupled to a distal portion of the catheter and adapted to couple to a rectum and prevent the catheter from exiting the anal sphincter.

11. The device of claim 10, wherein the distal retention element is a balloon.

12. The device of claim 10, wherein the sensor is coupled to the catheter, proximal to the distal retention element.

13. The device of claim 9, further comprising a proximal retention element, coupled to a proximal portion of the catheter and adapted to couple to an exterior portion of the patient and prevent the sensor from entering a rectum.

14. The device of claim 9, wherein the sensor comprises a plurality of sensors adapted to sense temperature at a plurality of locations within the anal sphincter.

15. The device of claim 9, further comprising a second sensor coupled to the catheter and adapted to sense intraperitoneal pressure.

16. The device of claim 15, further comprising a distal retention element, coupled to a distal portion of the catheter and adapted to couple to a rectum and prevent the catheter from exiting the anal sphincter, and wherein the sensor is coupled to the catheter, proximal to the distal retention element and the second sensor is coupled to the distal retention element.

17. A device for monitoring core temperature of a patient receiving intraperitoneal hypothermia or hyperthermia, the device comprising:
an airway management mechanism adapted to sample exhaled air from an airway of the patient;
a sensor coupled to the device and adapted to sense core temperature.

18. The device of claim 17 wherein the airway management mechanism comprises an endotracheal tube.

19. The device of claim 17 wherein the airway management mechanism comprises a catheter.

20. The device of claim 17 wherein the airway management mechanism comprises a bronchoscope.

21. The device of claim 17 wherein the airway management mechanism comprises a face mask.

22. The device of claim 17 wherein the airway management mechanism comprises a ventilation tube.

23. The device of claim 17, wherein the sensor is adapted to sense the temperature of exhaled air.

24. The device of claim 17, further comprising a distal retention element, coupled to a distal portion of the airway management mechanism and adapted to couple to the airway and prevent the airway management mechanism from moving.

25. The device of claim 24, wherein the distal retention element is a balloon.

26. The device of claim 24, wherein the sensor is coupled to the distal retention element.

27. The device of claim 17, wherein the sensor comprises a plurality of sensors adapted to sense temperature at a plurality of locations within the airway.

28. A device for monitoring core temperature of a patient receiving intraperitoneal hypothermia or hyperthermia, the device comprising:
a catheter adapted to be inserted into a non-intraperitoneal structure of the patient;
a plurality of sensors coupled to the catheter and adapted to sense temperature at a plurality of locations within the non-intraperitoneal structure; and
a processor coupled to the plurality of sensors and adapted to determine core temperature from temperatures sensed by the plurality of sensors.

29. The device of claim 28, wherein the processor is further adapted to select the portion of the temperatures sensed by the plurality of sensors having the highest temperatures.

30. The device of claim 28, wherein the processor is further adapted to select the portion of the temperatures sensed by the plurality of sensors having the lowest temperatures.

31. The device of claim 28, wherein the processor is further adapted to select the portion of the temperatures sensed by the plurality of sensors having a temperature that falls within a predetermined range.

32. The device of claim 28, wherein the catheter is adapted to be inserted into an esophagus.

33. The device of claim 28 wherein the catheter is adapted to be inserted into a urethra.

34. The device of claim 28, wherein the catheter is adapted to be inserted into an anal sphincter.

35. The device of claim 28, further comprising a heating and cooling device, coupled to the processor, and adapted to adjust the level of intraperitoneal hypothermia or hyperthermia given to the patient based on the core temperature determined by the processor.

36. The device of claim 28, further comprising:
a pressure sensor adapted to be inserted into an intraperitoneal structure of the patient, wherein the pressure sensor is coupled to the catheter and is coupled to the processor.

37. The device of claim 36, wherein the catheter is adapted to be inserted into an esophagus and the pressure sensor is adapted to be inserted into a stomach.

38. The device of claim 36, wherein the catheter is adapted to be inserted into a urethra and the pressure sensor is adapted to be inserted into a bladder.

39. The device of claim 36, wherein the catheter is adapted to be inserted into an anal sphincter and the pressure sensor is adapted to be inserted into a rectum.

40. A method of monitoring core temperature of a patient receiving intraperitoneal hypothermia or hyperthermia, the method comprising:
placing a monitoring device having a sensor into a urethra of the patient; and
sensing core temperature with the sensor.

41. The method of claim 40, wherein the step of placing a monitoring device comprises placing a plurality of sensors into the urethra of the patient.

42. The method of claim 41, wherein the step of sensing core temperature comprises sensing temperature at a plurality of locations within the urethra.

43. The method of claim 40, wherein the step of placing a monitoring device having a sensor comprises placing a monitoring device having a first sensor and a second sensor, the method further comprising:
placing the second sensor into a bladder; and
sensing an intraperitoneal parameter with the second sensor.

44. The method of claim 43, wherein the step of sensing the intraperitoneal parameter comprises sensing pressure.

45. A method of monitoring core temperature of a patient receiving intraperitoneal hypothermia or hyperthermia, the method comprising:
placing a monitoring device having a sensor into an anal sphincter of the patient; and
sensing core temperature with the sensor.

46. The method of claim 45, wherein the step of placing a monitoring device comprises placing a plurality of sensors into the anal sphincter of the patient.

47. The method of claim 46, wherein the step of sensing core temperature comprises sensing temperature at a plurality of locations within the anal sphincter.

48. The method of claim 45, wherein the step of placing a monitoring device having a sensor comprises placing a monitoring device having a first sensor and a second sensor, the method further comprising:
placing the second sensor into a rectum; and
sensing an intraperitoneal parameter with the second sensor.

49. The method of claim 48, wherein the step of sensing the intraperitoneal parameter comprises sensing pressure.

50. A method of monitoring core temperature of a patient receiving intraperitoneal hypothermia or hyperthermia, the method comprising:
placing a monitoring device having a sensor into an airway of the patient; and
sensing core temperature with the sensor.

51. The method of claim 50, wherein the step of sensing core temperature with the sensor comprises sensing the temperature of exhaled air.

52. The method of claim 50, wherein the step of placing a monitoring device comprises placing a plurality of sensors into the airway of the patient.

53. The method of claim 52, wherein the step of sensing core temperature comprises sensing temperature at a plurality of locations within the airway.

54. A method of monitoring core temperature of a patient receiving intraperitoneal hypothermia or hyperthermia, the method comprising:
placing a monitoring device having a plurality of sensors into a non-intraperitoneal structure of the patient;
sensing temperature with the plurality of sensors; and
determining core temperature from a portion of the temperatures sensed by the plurality of sensors.

55. The method of claim 54, wherein the step of determining core temperature comprises selecting a portion of the temperatures sensed by the plurality of sensors.

56. The method of claim 55, wherein the step of determining core temperature comprises selecting the highest sensed temperature.

57. The method of claim 55, wherein the step of determining core temperature comprises selecting the lowest sensed temperature.

58. The method of claim 55, wherein the step of determining core temperature comprises selecting temperatures sensed by the plurality of sensors that fall within a predetermined range.

59. The method of claim 54, wherein the step of placing a monitoring device comprises placing a monitoring device into an esophagus.

60. The method of claim 59, wherein the step of sensing temperature comprises sensing a temperature of the esophagus at a plurality of locations along the esophagus.

61. The method of claim 54, wherein the step of placing a monitoring device comprises placing a monitoring device into a urethra.

62. The method of claim 61, wherein the step of sensing temperature comprises sensing a temperature of the urethra at a plurality of locations along the urethra.

63. The method of claim 54, wherein the step of placing a monitoring device comprises placing a monitoring device into an anal sphincter.

64. The method of claim 63, wherein the step of sensing temperature comprises sensing a temperature of the anal sphincter at a plurality of locations along the anal sphincter.

65. The method of claim 54, further comprising the step of adjusting the level of intraperitoneal hypothermia or hyperthermia given to the patient based on the core temperature determined from a portion of the temperatures sensed by the plurality of sensors.

66. The method of claim 54 further comprising the steps of:
placing a sensor into an intraperitoneal structure;
sensing a parameter with the sensor; and
determining an intraperitoneal parameter from the parameter sensed by the sensor in the intraperitoneal structure.

67. The method of claim 66, wherein the step of determining the intraperitoneal parameter comprises determining pressure.

68. The method of claim 66, wherein the step of placing a monitoring device comprises placing a monitoring device into an esophagus and the step of placing a sensor into an intraperitoneal structure comprises placing a sensor into a stomach.

69. The method of claim 66, wherein the step of placing a monitoring device comprises placing a monitoring device into a urethra and the step of placing a sensor into an intraperitoneal structure comprises placing a sensor into a bladder.

70. The method of claim 66, wherein the step of placing a monitoring device comprises placing a monitoring device into an anal sphincter and the step of placing a sensor into an intraperitoneal structure comprises placing a sensor into a rectum.
